(19) United States
(12) Patent Application Publication
(10) Pub. No.: US 2024/0100012 A1
(43) Pub. Date: Mar. 28, 2024

(54) PHARMACEUTICAL DOSAGE FORM

(71) Applicant: Mark HASLETON, Raanana (IL)

(72) Inventors: Anton FRENKEL, Raanana (IL); Mark HASLETON, Raanana (IL)

(21) Appl. No.: 18/276,295

(22) PCT Filed: Jan. 17, 2022
(86) PCT No.: PCT/IB2022/050342

§ 371 Date: Aug. 8, 2023

Related U.S. Application Data
(60) Provisional application No. 63/138,529, filed on Jan. 18, 2021.

(51) Int. Cl. A61K 31/343; A61K 9/00

(52) U.S. Cl. CPC A61K 31/343; A61K 9/0019

(57) ABSTRACT

There are disclosed are pharmaceutical dosage forms comprising escitalopram base, as well as the use of the dosage forms in the treatment of certain conditions. Other embodiments are also disclosed.

### BACKGROUND

Depression is a common illness worldwide, with more than 250 million sufferers. It affects an estimated one in fifteen adults in any given year and one in six people will experience depression at some time in their life. Especially when long-lasting and with moderate or severe intensity, depression may become a serious health condition. It can cause the affected person to suffer greatly and function poorly at work, at school and in the family. At its worst, depression can lead to suicide. Close to 800,000 people die due to suicide every year and suicide remains the second leading cause of death in 15-29 year-olds. A recent study showed an increase in the diagnosis of major depressive disorder in the US from 6% in 1996 to over 10% in 2015. The same study showed that only 70% of patients received any antidepressant therapy.

The most common treatment of depression for more than twenty years has been the oral administration of antidepressants such as serotonin reuptake inhibitors. Current estimates are that this drug class accounts for between 70 and 90% of all US antidepressant prescriptions.

Antidepressants are also among the most commonly prescribed classes of drugs in Europe and the US, with the number of prescriptions and duration of use rising year on year. Between 2000 and 2018, the UK has experienced a 170% rise in their usage with an estimation that over seven million adults in England (16% of the adult population) were being prescribed an antidepressant in 2017 and over half of these patients, had been taking the medications for longer than two years. Similar numbers can be seen in the US, where usage has risen from 8% of the population in 2002 to almost 13% (37 million adults) by 2014 with around half of these patients taking the drugs for at least five years. In both the UK and US, the average duration of antidepressant usage has also more than doubled in the ten years between 2005 and 2015.

Despite the wide range of available options and the extensive prescribing of antidepressants, a study of adherence data from over 740,000 newly initiated immediate-release SSRI patients found that nearly 50% of patients failed to adhere to therapy for a minimum of 60 days, and only 28% were compliant at 6 months. There therefore remains a great need for further treatment options of serotonin reuptake inhibitors which could improve patient compliance and treatment adherence.

### SUMMARY OF THE INVENTION

The present invention relates to sustained release injectable pharmaceutical dosage forms comprising escitalopram base.

### DETAILED DESCRIPTION OF THE INVENTION

In the present disclosure the singular forms ‘a,’ ‘an,’ and ‘the’ include the plural reference, and reference to a particular numerical value includes at least that particular value, unless the context clearly indicates otherwise. Thus, for example, a reference to ‘a compound’ is a reference to one or more of such compounds and equivalents thereof known to those skilled in the art, and so forth. The term ‘plurality’, as used herein, means more than one. When a range of values is expressed, another embodiment includes from the one particular and/or to the other particular value. Similarly, when values are expressed as approximations, by use of the antecedent ‘about,’ it is understood that the particular value forms another embodiment. All ranges are inclusive and combinable.

When values are expressed as approximations, by use of the antecedent ‘about,’ it will be understood that the particular value forms another embodiment. As used herein, ‘about X’ (where X is a numerical value) preferably refers to ±10% of the recited value, inclusive. For example, the phrase ‘about 8’ refers to a value of 7.2 to 8.8, inclusive; as another example, the phrase ‘about 8%’ refers to a value of 7.2% to 8.8%, inclusive. Where present, all ranges are inclusive and combinable. For example, when a range of ‘1 to 5’ is recited, the recited range should be construed as including ranges ‘1 to 4’, ‘1 to 3’, ‘1 to 2’, ‘1 to 2 and 4 to 5’, ‘1 to 3 and 5’, and the like. In addition, when a list of alternatives is positively provided, such a listing can also include embodiments where any of the alternatives may be excluded. For example, when a range of ‘1 to 5’ is described, such a description can support situations whereby any of 1, 2, 3, 4, or 5 are excluded; thus, a recitation of ‘1 to 5’ may support ‘1 and 3-5, but not 2’, or simply ‘wherein 2 is not included.’

As used herein, the terms ‘component,’ ‘composition,’ ‘composition of compounds,’ ‘compound,’ ‘drug,’ ‘pharmacologically active agent,’ ‘active agent,’ ‘therapeutic,’ ‘therapy,’ ‘treatment,’ or ‘medicament’ are used interchangeably herein to refer to a compound or compounds or composition of matter which, when administered to a human subject induces a desired pharmacological and/or physiologic effect by local and/or systemic action.

As employed above and throughout the disclosure the term ‘effective amount’ refers to an amount effective, at dosages, and for periods of time necessary, to achieve the desired result with respect to the treatment of the relevant disorder, condition, or side effect. It will be appreciated that the effective amount of components of the present invention will vary from patient to patient not only with respect to the particular compound, component or composition selected, the route of administration, and the ability of the components to elicit a desired result in the individual, but also with respect to factors such as the disease state or severity of the condition to be alleviated, hormone levels, age, sex, weight of the individual, metabolic rate of the individual, the state of being of the patient, and the severity of the pathological condition being treated, concurrent medication or special diets then being followed by the particular patient, and other factors which those skilled in the art will recognize, with the appropriate dosage being at the discretion of the attending physician. Dosage regimes may be adjusted to provide improved therapeutic response. An effective amount is also one in which any toxic or detrimental effects of the components are outweighed by the therapeutically beneficial effects.

The present invention relates to a sustained release injectable pharmaceutical dosage form comprising escitalopram base.

As used herein the term ‘sustained release injectable pharmaceutical dosage form’ refers to an injectable dosage form that provides for the gradual release of escitalopram into the bloodstream over a period of time that is preferably at least 21 days.

The pharmaceutical dosage forms of the invention encompass dosage forms that are suitable for use with humans without undue toxic side effects. Dosage forms within the scope of the invention include the active pharmaceutical ingredient, escitalopram, and at least one pharmaceutically acceptable carrier or excipient. Examples of pharmaceutical dosage forms of the invention include, for example, microcapsules, nanocapsules, microspheres, nanospheres, microparticles, nanoparticles, polymer-drug conjugates, micelles, liposomes, hydrogels and other in-situ forming depots or implants. Said dosage forms can be formulated using biodegradable polymers or other suitable materials using methods known in the art.

Examples of biodegradable polymers useful for preparing the dosage forms of the disclosure include poly(lactide), poly(glycolide), poly(lactide-co-glycolide), poly-1-lactic acid, poly-d-lactic acid, poly(glycolic acid), copolymers of the foregoing, poly(aliphatic carboxylic acids), copolyoxalates, polycaprolactone, polydioxanone, poly(ortho carbonates), poly(acetals), poly(lactic acid-caprolactone), polyorthoesters, poly(glycolic acid-captolactone), poly(amino acid), polyesteramide, polyanhydrides, polyphosphazines, poly(alkylene alkylate), biodegradable polyurethane, polyvinylpyrrolidone, polyalkanoic acid, polyethylene glycol, copolymer of polyethylene glycol and polyorthoester, albumin, chitosan, casein, waxes or blends or copolymers thereof.

Examples of platform technologies that are useful in preparing the sustained release pharmaceutical dosage forms of the present disclosure include those associated with Novartis (see, e.g., WO2010018159), Alkermes (see, e.g., WO200191720), Allergan (see, e.g., WO2013112434), Reckitt Benckiser (see, e.g., WO2009091737), Icon Bioscience (see, e.g., WO2013036309), Flamel Technologies (see, e.g., WO2012080986), QLT (see, e.g., WO2008153611), Rovi Pharmaceuticals (see, e.g., WO2011151356), Dong-A (see, e.g., WO2008130158), Durect (see, e.g., WO2004052336), NuPathe (see, e.g., WO2005070332), Ascendis Pharma (see, e.g., WO2011042453), Endo (see, e.g., WO2013063125), Delpor (see, e.g., WO2010105093), PolyActiva (see, e.g., WO2010040188), Flexion Therapeutics (see, e.g., WO2012019009), pSivida (see, e.g., WO2005002625), Camurus (see, e.g., WO2005117830), Bind Therapeutics (see, e.g., WO2010075072), Zogenix (see, e.g., WO2007041410), Ingell (WO2011083086), Foresee Pharmaceuticals (see, e.g., WO2008008363), Medincell (see, e.g., WO2012090070), Mapi Pharma (see, e.g., WO2011080733), DelSiTech (see, e.g., WO2008104635), OctoPlus (see, e.g., WO2005087201), Nanomi (see, e.g., WO2005115599), Peptron (see, e.g., WO2008117927), GP Pharm (see, e.g., WO2009068708), Pharmathen (see, e.g., WO2014202214), Titan Pharmaceuticals (see, e.g., WO2007139744), Tolmar (see, e.g., WO2009148580), Heron Therapeutics (see, e.g., US2014323517) and Intarcia Therapeutics (see, e.g., WO2005048952). The disclosures of each of these published international patent applications are incorporated herein by reference in their entireties. Methods for formulating an active ingredient, or a pharmaceutically acceptable salt thereof, into a dosage form of suitable for use in the instant methods are also described in, for example, Hu et al., IJPSR, 2012; vol. 3(9): 2888-2896; Hoffman, Adv. Drug. Del. Rev. 54 (2002) 3-12; Al-Tahami et al. Recent Patents on Drug Del. & Formulation 2007, 1 65-71; Pattni et al. Chem. Rev. 2015 May 26; and Wright and Burgess (ed.) Long Acting Injections and Implants (2012), the disclosures of which are incorporated herein by reference in their entireties.

As used herein, the term ‘escitalopram’ or ‘escitalopram base’ shall refer to the compound S-1-[3-(dimethylamino)propyl]-1-(4-fluorophenyl)-1,3-dihydro-5-isobenzofurancarbonitrile and hydrates and solvates thereof.

As used herein, the term ‘hydrate’ refers to escitalopram base having a stoichiometric or non-stoichiometric amount of water bound by non-covalent intermolecular forces, for example, the hydrated form of escitalopram base. The hydrate may comprise at least one equivalent of water, for example, one to five equivalents of water. It may be prepared by crystallizing the compounds, or pharmaceutically acceptable salt thereof, in water or an aqueous solvent.

As used herein, the term ‘solvate’ refers to escitalopram base having a stoichiometric or non-stoichiometric amount of a solvent bound by non-covalent intermolecular forces. In a preferred embodiment, the solvent is non-volatile, non-toxic, and suitable for administration to humans including, for example, ethanol, methanol, propanol, isopropanol and methylene chloride.

In one embodiment of the invention, the escitalopram base is crystalline. In another embodiment of the invention, the escitalopram base is an oil. In one embodiment of the invention, the crystalline escitalopram base is a hydrate. In another embodiment of the invention, the crystalline escitalopram base is a solvate. In another embodiment of the invention, the escitalopram base is mixed with 0.5-5% of a solvent. In a preferred embodiment of the invention, the crystalline escitalopram base is an isopropanolate.

Remarkably, it has now been identified that due to their high solubility in organic solvents and low aqueous solubility, crystalline and/or oil forms of escitalopram base are ideal SSRI antidepressants to be formulated in sustained release injectable pharmaceutical dosage forms. Escitalopram base, whether crystalline or oil, is found to demonstrate extremely high solubility in organic solvents, such as N-Methyl-2-pyrrolidone (NMP) and Dimethyl sulfoxide (DMSO) making it an ideal drug product for sustained release injectable pharmaceutical dosage forms. Solutions of oil forms of escitalopram base are found to especially demonstrate extremely high solubility of the escitalopram in organic solvents and solutions with more than 70% wt of escitalopram can be prepared.

In one embodiment of the invention, the escitalopram base is an oil and comprises less than about 0.5% of any residual solvent. In another embodiment of the invention, the escitalopram base is an oil and is substantially free of any residual solvent. As used herein, the term “substantially free” shall refer to less than about 0.1%. In one embodiment of the invention, the escitalopram base as an oil comprising less than 0.5% of any residual solvent, is prevented from recrystallizing by the addition of 0.5-5% of an additional solvent such as ethanol, glycerin, propylene glycol and/or polyethylene glycol.

In one embodiment of the invention, the escitalopram base is co-formulated together with an organic solvent and an acid excipient prior to its introduction to the remaining excipients of the sustained release injectable pharmaceutical dosage form. In one embodiment of the invention, the co-formulation of escitalopram base, solvent and acid lead to the formation of an in-situ salt of escitalopram. In one embodiment of the invention, the acid excipient is glycolic acid. In another embodiment of the invention, the acid excipient is acetic acid. In one embodiment of the invention, an equimolar amount of the acid excipient is co-formulated together with the escitalopram base. In another embodiment of the invention, a less than equimolar amount of the acid excipient is co-formulated together with the escitalopram base.

In one embodiment of the invention, the sustained release injectable pharmaceutical dosage form comprises escitalopram base and an organic solvent. In one preferred embodiment of the invention, the organic solvent is NMP. In another preferred embodiment of the invention, the preferred solvent is DMSO.

In one embodiment of the invention, the sustained release injectable pharmaceutical dosage form comprises escitalopram base, an organic solvent and at least one biodegradable polymer. In one embodiment, the at least one biodegradable is selected from the group comprising poly(lactide), poly(glycolide), poly(lactide-co-glycolide), poly-1-lactic acid, poly-d-lactic acid, poly(glycolic acid), copolymers of the foregoing, poly(aliphatic carboxylic acids), copolyoxalates, polycaprolactone, polydioxanone, poly(ortho carbonates), poly(acetals), poly(lactic acid-caprolactone), polyorthoesters, poly(glycolic acid-captolactone), poly(amino acid), polyesteramide, polyanhydrides, polyphosphazines, poly(alkylene alkylate), biodegradable polyurethane, polyvinylpyrrolidone, polyalkanoic acid, polyethylene glycol, copolymer of polyethylene glycol and polyorthoester, albumin, chitosan, casein, waxes or blends or copolymers thereof. In one preferred embodiment of the invention, the organic solvent is NMP. In another preferred embodiment of the invention, the preferred solvent is DMSO. In one preferred embodiment of the invention, the sustained release injectable pharmaceutical dosage form comprises escitalopram base, DMSO and poly(lactide), poly(glycolide), poly(lactide-co-glycolide), poly-1-lactic acid, poly-d-lactic acid, poly(glycolic acid), polycaprolactone or copolymers thereof. In another preferred embodiment of the invention, the sustained release injectable pharmaceutical dosage form comprises escitalopram base, NMP and poly(lactide), poly(glycolide), poly(lactide-co-glycolide), poly-1-lactic acid, poly-d-lactic acid, poly(glycolic acid), polycaprolactone or copolymers thereof.

The escitalopram base of the present invention can be manufactured by methods know to those in the art and include those described in EP347066, US20040167209 and U.S. Pat. No. 7,723,533 the entirety of each of which are incorporated herein by reference.

Sustained release injectable pharmaceutical dosage form technologies are typically constrained by two features, the maximal volume that can be administered to a subject in need thereof and the volume of excipients necessary to achieve effective sustained release. These constraints leave minimal available volume within the dosage form for an active ingredient. Administering escitalopram base within a sustained release injectable pharmaceutical dosage form provides the further advantages of utilizing the SSRI with the lowest therapeutic dose and, by formulating the base rather than salt form of the compound, minimizing the necessary volume of API within the dosage form.

In one embodiment of the invention, the sustained release injectable pharmaceutical dosage form comprises at least 300 mg of escitalopram base. In another embodiment of the invention, the sustained release injectable pharmaceutical dosage form comprises at least 325 mg, at least 350 mg, at least 375 mg, at least 400 mg, at least 425 mg, at least 450 mg, at least 475 mg, at least 500 mg, at least 525 mg, at least 550 mg, at least 575 mg, at least 600 mg, at least 625 mg, at least 650 mg, at least 675 mg, at least 700 mg, at least 725 mg, at least 750 mg, at least 775 mg, at least 800 mg, at least 825 mg, at least 850 mg, at least 875 mg, at least 900 mg, at least 925 mg, at least 950 mg, at least 975 mg or at least 1000 mg of escitalopram base.

In one embodiment of the invention, the administration of escitalopram base comprises the administration of a sustained release injectable pharmaceutical dosage form comprising escitalopram base. In a preferred embodiment of the invention, the sustained release injectable pharmaceutical dosage form comprising escitalopram base is subcutaneously administered. In another preferred embodiment of the invention, the sustained release injectable pharmaceutical dosage form comprising escitalopram base is intramuscularly administered. In one embodiment of the invention, the administration of a sustained release injectable pharmaceutical dosage form comprising escitalopram base provides for the release of therapeutically effective amounts of escitalopram into the bloodstream.

In one embodiment of the invention, therapeutically effective amounts of escitalopram in the bloodstream are provided by the administration of a single dose of escitalopram base. In another embodiment of the invention, therapeutically effective amounts of escitalopram in the bloodstream are provided by the administration of multiple doses of escitalopram base. In one embodiment of the invention, therapeutically effective amounts of escitalopram in the bloodstream are provided by multiple doses of escitalopram base administered at least fourteen days apart from each other. In another embodiment of the invention, therapeutically effective amounts of escitalopram in the bloodstream are provided by multiple doses of escitalopram base administered at least twenty-eight days apart from each other. As used herein, the term ‘dose of escitalopram base’ shall refer to a single or multiple, individual, administrations of escitalopram base all within a six-hour time period.

In one embodiment of the invention, blood plasma levels of escitalopram rise after administration of escitalopram base to reach a maximum plasma level before gradually decreasing over time. In one embodiment of the invention, the plasma levels of escitalopram are maintained at a maximum by the administration of one or more further doses of escitalopram base before gradually decreasing over time.

In one embodiment of the invention, the administration of escitalopram base provides for therapeutically effective amounts of escitalopram in the bloodstream which gradually decrease over time until untraceable by standard analytical methodologies. In one embodiment of the invention, the gradual decrease of blood plasma escitalopram levels, after the administration of escitalopram base, occurs for a time period of fourteen days. In another embodiment of the invention, the gradual decrease of blood plasma escitalopram levels, after the administration of escitalopram base, occurs for a time period of twenty-eight days. In another embodiment of the invention, the gradual decrease of blood plasma escitalopram levels, after the administration of escitalopram base, occurs for a time period of thirty-five days. In another embodiment of the invention, the gradual decrease of blood plasma escitalopram levels, after the administration of escitalopram base, occurs for a time period of forty-two days. In another embodiment of the invention, the gradual decrease of blood plasma escitalopram levels, after the administration of escitalopram base, occurs for a time period of forty-nine days. In another embodiment of the invention, the gradual decrease of blood plasma escitalopram levels, after the administration of escitalopram base, occurs for a time period of fifty-six days. In another embodiment of the invention, the gradual decrease of blood plasma escitalopram levels, after the administration of escitalopram base, occurs for a time period of sixty-three days. In another embodiment of the invention, the gradual decrease of blood plasma escitalopram levels, after the administration of escitalopram base, occurs for a time period of seventy days. In another embodiment of the invention, the gradual decrease of blood plasma escitalopram levels, after the administration of escitalopram base, occurs for a time period of seventy-seven days. In another embodiment of the invention, the gradual decrease of blood plasma escitalopram levels, after the administration of escitalopram base, occurs for a time period of eighty-four days. In another embodiment of the invention, the gradual decrease of blood plasma escitalopram levels, after the administration of escitalopram base, occurs for a time period of four months. In another embodiment of the invention, the gradual decrease of blood plasma escitalopram levels, after the administration of escitalopram base, occurs for a time period of five months.

In one embodiment of the invention, the time for blood plasma levels of escitalopram to be untraceable by standard analytical methodologies is at least fourteen days, at least twenty-eight days, at least thirty-five days, at least forty-two days, at least forty-nine days, at least fifty-six days, at least sixty-three days, at least seventy days, at least seventy-seven days, at least eighty-four days, at least four months or at least five months after administration of the single or after the concluding dose of escitalopram base.

In one embodiment of the invention, the subject's blood plasma levels of escitalopram rise to a steady state of about 20 ng/ml during the time period after the first administration of escitalopram base. In another embodiment of the invention, the subject's blood plasma levels of escitalopram rise to a steady state of at least 20 ng/ml during the time period after the first administration of escitalopram base.

As used herein, the term ‘steady state’ or ‘steady state blood plasma levels’ refers to consistent blood plasma levels over a time period of at least three days.

In one embodiment of the invention, the subject maintains steady state blood plasma levels of escitalopram of about 20 ng/ml for at least three days after administration of escitalopram base. In another embodiment of the invention, the subject maintains steady state blood plasma levels of escitalopram of about 20 ng/ml for at least seven days after administration of escitalopram base. In another embodiment of the invention, the subject maintains steady state blood plasma levels of escitalopram of about 20 ng/ml for at least fourteen days after administration of escitalopram base. In another embodiment of the invention, the subject maintains steady state blood plasma levels of escitalopram of about 20 ng/ml for at least twenty-one days after administration of escitalopram base. In another embodiment of the invention, the subject maintains steady state blood plasma levels of escitalopram of about 20 ng/ml for at least twenty-eight days after administration of escitalopram base.

In one embodiment of the invention, the subject maintains steady state blood plasma levels of escitalopram of about 20 ng/ml for at least three days after injection of escitalopram base. In another embodiment of the invention, the subject maintains steady state blood plasma levels of escitalopram of about 20 ng/ml for at least seven days after injection of escitalopram base. In another embodiment of the invention, the subject maintains steady state blood plasma levels of escitalopram of about 20 ng/ml for at least fourteen days after injection of escitalopram base. In another embodiment of the invention, the subject maintains steady state blood plasma levels of escitalopram of about 20 ng/ml for at least twenty-one days after injection of escitalopram base. In another embodiment of the invention, the subject maintains steady state blood plasma levels of escitalopram of about 20 ng/ml for at least twenty-eight days after injection of escitalopram base.

In one embodiment of the invention, the subject maintains steady state blood plasma levels of escitalopram of at least 20 ng/ml for at least three days after administration of escitalopram base. In another embodiment of the invention, the subject maintains steady state blood plasma levels of escitalopram of at least 20 ng/ml for at least seven days after administration of escitalopram base. In another embodiment of the invention, the subject maintains steady state blood plasma levels of escitalopram of at least 20 ng/ml for at least fourteen days after administration of escitalopram base. In another embodiment of the invention, the subject maintains steady state blood plasma levels of escitalopram of at least 20 ng/ml for at least twenty-one days after administration of escitalopram base. In another embodiment of the invention, the subject maintains steady state blood plasma levels of escitalopram of at least 20 ng/ml for at least twenty-eight days after administration of escitalopram base.

In one embodiment of the invention, the subject maintains steady state blood plasma levels of escitalopram of at least 20 ng/ml for at least three days after injection of escitalopram base. In another embodiment of the invention, the subject maintains steady state blood plasma levels of escitalopram of at least 20 ng/ml for at least seven days after injection of escitalopram base. In another embodiment of the invention, the subject maintains steady state blood plasma levels of escitalopram of at least 20 ng/ml for at least fourteen days after injection of escitalopram base. In another embodiment of the invention, the subject maintains steady state blood plasma levels of escitalopram of at least 20 ng/ml for at least twenty-one days after injection of escitalopram base. In another embodiment of the invention, the subject maintains steady state blood plasma levels of escitalopram of at least 20 ng/ml for at least twenty-eight days after injection of escitalopram base.

In one embodiment of the invention, the subject achieves steady state blood plasma levels of escitalopram of about 20 ng/ml within 24 hours of injection of escitalopram base. In another embodiment of the invention, the subject achieves steady state blood plasma levels of escitalopram of about 20 ng/ml within 48 hours of injection of escitalopram base. In another embodiment of the invention, the subject achieves steady state blood plasma levels of escitalopram of about 20 ng/ml within 72 hours of injection of escitalopram base. In another embodiment of the invention, the subject achieves steady state blood plasma levels of escitalopram of about 20 ng/ml within 96 hours of injection of escitalopram base. In another embodiment of the invention, the subject achieves steady state blood plasma levels of escitalopram of about 20 ng/ml within 120 hours of injection of escitalopram base. In another embodiment of the invention, the subject achieves steady state blood plasma levels of escitalopram of about 20 ng/ml within 144 hours of injection of escitalopram base. In another embodiment of the invention, the subject achieves steady state blood plasma levels of escitalopram of about 20 ng/ml within 168 hours of injection of escitalopram base.

In one embodiment of the invention, the subject achieves steady state blood plasma levels of escitalopram of at least 20 ng/ml within 24 hours of injection of escitalopram base. In another embodiment of the invention, the subject achieves steady state blood plasma levels of escitalopram of at least 20 ng/ml within 48 hours of injection of escitalopram base. In another embodiment of the invention, the subject achieves steady state blood plasma levels of escitalopram of at least 20 ng/ml within 72 hours of injection of escitalopram base. In another embodiment of the invention, the subject achieves steady state blood plasma levels of escitalopram of at least 20 ng/ml within 96 hours of injection of escitalopram base. In another embodiment of the invention, the subject achieves steady state blood plasma levels of escitalopram of at least 20 ng/ml within 120 hours of injection of escitalopram base. In another embodiment of the invention, the subject achieves steady state blood plasma levels of escitalopram of at least 20 ng/ml within 144 hours of injection of escitalopram base. In another embodiment of the invention, the subject achieves steady state blood plasma levels of escitalopram of at least 20 ng/ml within 168 hours of injection of escitalopram base.

In one embodiment of the invention, the sustained release injectable pharmaceutical dosage form comprising escitalopram base are administered to a human subject in need thereof. In one embodiment of the invention, the administration of the sustained release injectable pharmaceutical dosage form comprising escitalopram base is to treat a psychiatric condition. In one embodiment of the invention, the psychiatric condition is Major Depressive Disorder (MDD). In another embodiment of the invention, the psychiatric condition is anxiety. In one embodiment of the invention, the anxiety is Generalized Anxiety Disorder. In another embodiment of the invention, the anxiety is Social Anxiety Disorder.

This invention will be better understood by reference to the Examples, which follow, but those skilled in the art will readily appreciate that the specific experiments detailed are only illustrative of the invention as described more fully in the claims which follow thereafter.

### EXAMPLES

### Example 1: Preparation of Escitalopram Base from Escitalopram Oxalate Salt

Escitalopram oxalate (40 g) and deionized water (170 ml) were introduced to a 250 ml jacketed glass reactor equipped with a mechanical stirrer, circulating oil bath and thermometer. While the mixture was stirred, 45 ml of ether was added with the jacket temperature maintained at 25° C. throughout the isolation procedure. The pH of the mixture was adjusted to 9.0-9.5 by the addition of 25% NH 4 OH. The stirrer was stopped to allow the settling of the mixture. Two liquid phases and solid precipitates formed. The resultant mixture was filtered and the obtained solid cake washed with 40 ml of ether. The filtrate and ether wash were then re-introduced into the reactor. Organic and aqueous phases were separated and collected into different containers. The aqueous phase was re-introduced to the reactor and extracted with 50 ml of ether. After settling, the aqueous phase was discarded. The two organic extracts were mixed in the reactor and washed twice with 25 ml of water. The organic solution was evaporated in a rotary evaporator under vacuum, with the bath temperature maintained at 70° C., until complete evaporation of solvent occurred. The resultant escitalopram base residue, 30.1 g of colorless clear oil (hot) and semi-solid at room temperature, was transferred to an amber glass vial.

### Example 2: Solvent-Free Escitalopram Base

Escitalopram oxalate and 20-25 volumes of deionized water are charged to jacketed glass reactor equipped with a stirrer, thermometer and circulation bath for heating and cooling.

The mixture is heated to 70-100° C. and stirred until solid dissolution. Then the pH is adjusted to within the range 9-11 by addition of an aqueous base (KON, NaOH or NH₄OH) under heat. This adjustment leads to precipitation of liquid escitalopram free base. The batch is cooled by stirring, filtered for removal of precipitated solids and reintroduced to the reactor. The mixture is then stirred and settled for phase separation where the lower aqueous layer is separated and discarded and the upper organic layer washed twice with 2 volumes of deionized water to result in the collection of escitalopram free base as a viscous oil.

Those skilled in the art will appreciate that numerous changes and modifications can be made to the preferred embodiments of the disclosure and that such changes and modifications can be made without departing from the spirit of the disclosure. It is, therefore, intended that the appended claims cover all such equivalent variations as fall within the true spirit and scope of the disclosure.

We claim:

1. A sustained release injectable pharmaceutical dosage form comprising escitalopram base.

2. The dosage form of claim 1, wherein the escitalopram base is crystalline.

3. The dosage form of claim 1, wherein the escitalopram base is an oil.

4. The dosage form of any preceding claim, wherein the escitalopram base forms an in-situ salt within the dosage form with an acid excipient

5. The dosage form of claim 4, wherein the acid excipient is selected from the group consisting of glycolic and acetic acid.

6. The dosage form of any preceding claim further comprising an organic solvent.

7. The dosage form of claim 6, wherein the organic solvent comprises NMP or DMSO.

8. The dosage form of any preceding claim comprising at least 600 mg of escitalopram base.

9. A method of treating a psychiatric condition comprising administering, to a human subject in need thereof, the dosage form of any preceding claim.

10. The method of claim 9, wherein the condition treated is Major Depressive Disorder.

11. The method of claim 9, wherein the condition treated is Generalized Anxiety Disorder.

12. The method of claim 9, wherein the condition treated is Social Anxiety Disorder.

13. The method of claim 9, wherein the administration results in steady state plasma levels of escitalopram of at least 20 ng/ml within five days of the administration.

14. The method of claim 9, wherein the administration results in steady state plasma levels of escitalopram of at least 20 ng/ml for at least 21 days after the administration.
